(19) United States
(12) Patent Application Publication
(10) Pub. No.: US 2003/0040630 A1
(43) Pub. Date: Feb. 27, 2003

(54) Method for producing sulfonyl imidazole derivatives

(76) Inventors: Holger Weintritt, Langenfeld (DE); Reinhard Lantzsch, Wuppertal (DE); Thorsten Muh, Leverkusen (DE)

Correspondence Address: BAYER CORPORATION, PATENT DEPARTMENT, 100 BAYER ROAD, PITTSBURGH, PA, 15205, US

(21) Appl. No.: 10/182,965

(22) PCT Filed: Jan. 25, 2001
(86) PCT No.: PCT/EP01/00794

(30) Foreign Application Priority Data
Feb. 7, 2000 (DE) 100 05 278.9

(51) Int. Cl. C07D 233/52

(52) U.S. Cl. 548/316.7; 548/331.1; 548/311.1

(57) ABSTRACT

Sulphonyl-imidazole derivatives of the formula

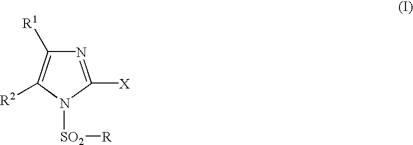

in which

R,R¹, R² and X are as defined in the description can be prepared by a new process by reacting imidazole derivatives of the formula

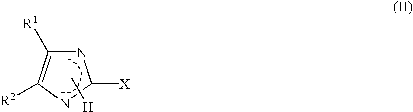

with sulphonyl chlorides of the formula

Cl—SO₂—R (III)

in the presence of an acid binder, of an organic diluent which is sparingly miscible with water and in the presence of water and a phase transfer catalyst.

### PROCESS FOR THE PREPARATION OF SULPHONYL-IMIDAZOLE DERIVATIVES

The present invention relates to a new process for the preparation of known sulphonyl-imidazole derivatives which can be used as active substances with microbicidal properties.

It has already been disclosed that sulphonyl-imidazole derivatives can be synthesized by reacting imidazole derivatives with sulphonyl halides in the presence of an acid binder and in the presence of a diluent (cf. WO 97-06 171, EP-A 0 893 445, DE-A 198 29 740, WO 97-47 620 and JP-A 54-90 175). Thus, for example, 1-(3,5-dimethylisoxazole-4-sulphonyl)-2-chloro-6,6-difluoro-[1,3]-dioxolo-[4,5-f]-benzimidazole is obtained by reacting 2-chloro-6,6-difluoro-[1,3]-dioxolo-[4,5-f]-benzimidazole with 3,5-dimethylisoxazolesulphonyl chloride in the presence of potassium carbonate and acetonitrile. However, the disadvantage of this process is that the yields of the desired products are relatively low, and an application of this process on an industrial scale is therefore unsatisfactory.

It has now been found that sulphonyl-imidazole derivatives of the formula

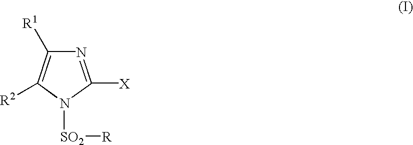

in which

R¹ and R² are identical or different and independently of one another represent hydrogen, halogen, cyano, thiocyanato, nitro, formyl, carboxyl, carbamoyl, thio-carbamoyl, alkyl, alkoxy, alkylthio, alkylsulphinyl, alkylsulphonyl, alkenyl, alkenyloxy, halogenoalkyl, halogenoalkoxy, halogenoalkylthio, halogenoalkyl-sulphinyl, halogenoalkylsulphonyl, halogenoalkenyl, halogenoalkenyloxy, alkylcarbonyl, alkylcarbonyloxy, alkoxycarbonyl, alkylsulphonyloxy, alkox-iminoalkyl, cycloalkyl, dialkylamino or Z-R³ where

Z represents a direct bond, an oxygen atom, alkanediyl, alkenediyl, alkinediyl, -*Q-CQ-, -*CQ-Q-, —*CH₂-Q-, -Q*-CH₂—, -*CQ-Q-CH₂—, —*CH₂-Q-CQ-, -*Q-CQ-CH₂—, -*Q-CQ-Q-CH₂—, —S(O)ₙ—, —*CH₂—S(O)ₙ—, -CQ- or -*S(O)ₙ—CH₂—, in which

Q represents oxygen or sulphur and the atoms marked (*) are in each case linked to R³,

n represents 0, 1 or 2 and

R³ represents optionally substituted aryl or optionally substituted heterocyclyl,

or

R¹ and R² together represent a divalent radical of the formula

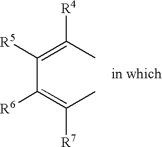

R⁴, R⁵, R⁶ and R⁷ independently of one another represent hydrogen, halogen, cyano, nitro, alkyl, halogenoalkyl, alkoxy, halogenoalkoxy, alkylthio, halo-genoalkylthio, alkylsulphinyl, halogenoalkylsulphinyl, alkylsulphonyl, halogenoalkylsulphonyl, optionally substituted cycloalkyl, hydroxycarbonyl, alkylcarbonyl, alkoxycarbonyl, cycloalkylcarbonyl, cycloalkoxycarbonyl or -Z¹-R⁸, in which

R⁸ represents optionally substituted aryl or optionally substituted heterocyclyl and

Z¹ represents a direct bond, or represents —CH₂—, O, S, SO, SO₂, or CO, or

represents —CO—O—, where the oxygen atom is linked to R⁸, or

represents —SO₂—O—, where the sulphur atom is linked to R⁸, or

represents —S—CH₂—SO₂—, where the sulphur atom of the thio group is linked to R⁸,

or

R⁴ and R⁵, or R⁵ and R⁶, or R⁶ and R⁷ in each case together represent an optionally substituted alkylene chain with 3 or 4 members in which one or two (non-adjacent) carbon atoms can be replaced by oxygen atoms,

R represents alkyl, halogenoalkyl, alkoxy, halogenoalkoxy, alkylthio, halogenoalkylthio, alkenyl, halogenoalkenyl, alkenyloxy, alkenylthio, alkinyl, alkinyloxy, alkinylthio, alkylamino, dialkylamino, optionally substituted aryl, optionally substituted aryloxy, optionally substituted arylthio, optionally substituted cycloalkyl, optionally substituted cycloalkyloxy, optionally substituted cycloalkylthio, optionally substituted cycloalkylamino, optionally substituted di-cycloalkyl-amino or represents an optionally substituted, saturated or unsaturated heterocyclic radical

and

X represents fluorine, chlorine or bromine, are obtained by reacting imidazole derivatives of the formula

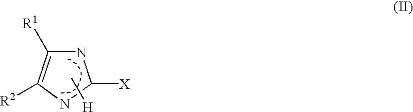

in which

R¹, R² and X are as defined above with sulphonyl chlorides of the formula

Cl—SO₂—R (III)

in which R is as defined above,

in the presence of an acid binder, of an organic diluent which is sparingly miscible with water and in the presence of water and a phase transfer catalyst.

It must be considered as extremely surprising that sulphonyl-imidazole derivatives of the formula (I) can be prepared by the process according to the invention in a smooth reaction without troublesome side reactions. Thus, it was to be expected on the basis of the known prior art that imidazole derivatives which have a halogen atom in the 2-position are hydrolysed at least to some extent in the presence of water and a phase transfer catalyst (cf. Houben-Weyl “Methoden der Organischen Chemie” [Methods in organic chemistry], Volume E8c, page 365, Georg Thieme Verlao, Stuttgart, New York 1994 and Ind. J. Chem. 26 B, (1987) 985-986). However, in contrast to what was expected, the reaction according to the invention is not adversely affected by such a hydrolysis. It is furthermore surprising that the target products are obtained in a markedly higher yield than by the methods known to date.

The process according to the invention is distinguished by a series of advantages. Thus, it allows sulphonyl-imidazole derivatives of the formula (I) to be synthesized in extremely high yield and outstanding purity. It is also advantageous that the procedure of the reaction and the isolation of the reaction products causes no difficulties whatsoever. Moreover, the process according to the invention can also be applied without problems at the industrial scale.

If 2-chloro-6,6-difluoro-[1,3]-dioxolo-[4,5-f]-benzimidazole and 3,5-dimethyl-isoxazole-4-sulphonyl chloride are employed as starting materials, potassium carbonate as acid binder, toluene in a mixture with water as diluent and tetra-(n-butyl)-ammonium bromide as phase transfer catalyst, the course of the process according to the invention can be illustrated by the following formulaic scheme.

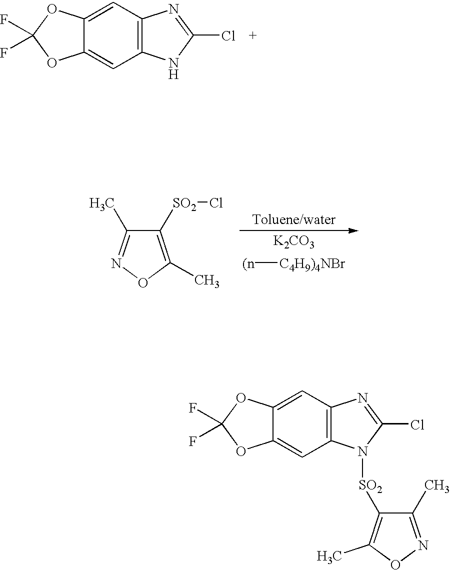

Formula(II) provides a general definition of the imidazole derivatives required as starting materials for carrying out the process according to the invention. These imidazole derivatives can exist in the following two tautomeric forms.

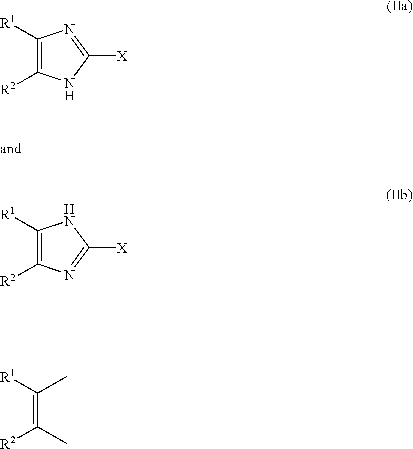

is symmetrical, the tautomeric forms are identical. In this case, the reaction according to the invention with sulphonyl chloride of the formula (III) therefore only yields one particular end product of the formula (I) in each case, independently of which of the two tautomeric forms reacts.

If the moiety

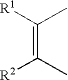

is asymmetrical, the two tautomeric forms are not identical. In this case, the reaction according to the invention with sulphonyl chloride of the formula (II) can therefore yield end products of the formula (I) which are derived from the tautomeric forms (IIa) and/or (IIb). If both tautomers react, end products of the formula (I) are obtained in the form of mixtures.

Preferred starting materials when carrying out the process according to the invention are imidazole derivatives of the formula (II) in which

R¹ and R² are identical or different and independently of one another represent hydrogen, fluorine, chlorine, bromine, iodine, cyano, thiocyanato, nitro, formyl, carboxyl, carbamoyl, thiocarbamoyl, alkyl having 1 to 4 carbon atoms, alkoxy having 1 to 4 carbon atoms, halogenoalkyl having 1 to 4 carbon atoms and 1 to 5 identical or different halogen atoms, halogenoalkoxy having 1 to 4 carbon atoms and 1 to 5 identical or different halogen atoms, alkoxycarbonyl having 1 to 4 carbon atoms in the alkoxy moiety, cycloalkyl having 3 to 6 carbon atoms, halogenoalkylsulphinyl having 1 to 4 carbon atoms and 1 to 5 identical or different halogen atoms, halogenoalkylsulphonyl having 1 to 4 carbon atoms and 1 to 5 identical or different halogen atoms, dialkylamino having 1 to 4 carbon atoms in each alkyl group, alkylcarbonyl having 1 to 4 carbon atoms in the alkyl moiety, alkoximinoalkyl having 1 to 4 carbon atoms in the alkoxy moiety and 1 to 4 carbon atoms in the alkyl moiety, alkylcarbonyloxy having 1 to 4 carbon atoms in the alkyl moiety, halogenoalkylcarbonyloxy having 1 to 4 carbon atoms in the halogenoalkyl group and 1 to 5 identical or different halogen atoms, alkylthio having 1 to 4 carbon atoms, alkylsulphinyl having 1 to 4 carbon atoms, alkylsulphonyl having 1 to 4 carbon atoms, alkenyl having 2 to 4 carbon atoms, alkenyloxy having 2 to 4 carbon atoms, halogenoalkylthio having 1 to 4 carbon atoms and 1 to 5 identical or different halogen atoms, halogenoalkenyl having 2 to 4 carbon atoms and 1 to 5 identical or different halogen atoms, halogenoalkenyloxy having 2 to 4 carbon atoms and 1 to 5 identical or different halogen atoms, alkylsulphonyloxy having 1 to 4 carbon atoms, or represent Z-R³

in which

Z represents a direct bond, an oxygen atom, alkanediyl having 1 to 4 carbon atoms, alkenediyl having 2 to 4 carbon atoms, alkinediyl having 2 to 4 carbon atoms or a group of the formula

-*Q-CQ-, -*CQ-Q-, —*CH₂-Q-, -*Q-CH₂—, -*CQ-Q-CH₂—, —*CH₂-Q-CQ-, -*Q-CQ-CH₂—, -*Q-CQ-Q-CH₂—, —S(O)ₙ—, —*CH₂—S(O)ₙ—, -CQ- or —*S(O)ₙ—CH₂—,

where in each case the atoms marked * are linked to R³.

Q represents oxygen or sulphur,

n represents 0, 1 or 2

and

R³ represents phenyl or naphthyl, it being possible for each of these two radicals to be monosubstituted to trisubstituted by identical or different substituents from the series consisting of halogen, cyano, nitro, formyl, carbamoyl, thiocarbamoyl, alkyl having 1 to 4 carbon atoms, alkoxy having 1 to 4 carbon atoms, alkylthio having 1 to 4 carbon atoms, alkenyl or alkenyloxy having in each case 2 to 4 carbon atoms, alkinyl or alkinyloxy having in each case 2 to 4 carbon atoms, halogenoalkyl, halogenoalkoxy or halogenoalkylthio having in each case 1 to 4 carbon atoms and 1 to 5 identical or different halogen atoms, halogenoalkenyl or halogenoalkenyloxy having in each case 2 to 4 carbon atoms and 1 to 5 identical or different halogen atoms, halogenoalkinyl or halogenoalkinyloxy having in each case 2 to 4 carbon atoms and 1 to 5 identical or different halogen atoms, dialkylamino, alkoxycarbonyl or alkoxirninoalkyl having in each case 1 to 4 carbon atoms in the individual alkyl moieties, cycloalkyl having 3 to 6 carbon atoms, alkylsulphinyl having 1 to 4 carbon atoms, alkylsulphonyl having 1 to 4 carbon atoms, halogenoalkylsulphinyl having 1 to 4 carbon atoms and 1 to 5 identical or different halogen atoms, halogenoalkylsulphonyl having 1 to 4 carbon atoms and 1 to 5 identical or different halogen atoms, alkylcarbonyl having 1 to 4 carbon atoms in the alkyl group, alkylcarbonyloxy having 1 to 4 carbon atoms in the alkyl moiety and/or alkylsulphonyloxy having 1 to 4 carbon atoms, or else can be monosubstituted by divalent dioxyalkylene which has 1 or 2 carbon atoms and which is optionally monosubstituted to tetrasubstituted by identical or different halogen substituents, the oxygen atoms not being adjacent,

or

R³ represents an unsaturated heterocyclyl radical having 5 or 6 ring members and 1 to 3 hetero atoms such as nitrogen, oxygen and/or sulphur, it being possible for these radicals to be monosubstituted to trisubstituted by identical or different substituents from the series consisting of halogen, alkyl having 1 to 4 carbon atoms, alkoxy having 1 to 4 carbon atoms, halogenoalkyl having 1 to 4 carbon atoms and 1 to 5 identical or different halogen atoms, halogenoalkoxy having 1 to 4 carbon atoms and 1 to 5 identical or different halogen atoms, alkoxycarbonyl having 1 to 3 carbon atoms in the alkoxy moiety, cycloalkyl having 3 to 6 carbon atoms, cyano and/or nitro,

and

X represents chlorine or bromine.

Other preferred starting materials for carrying out the process according to the invention are imidazole derivatives of the formula

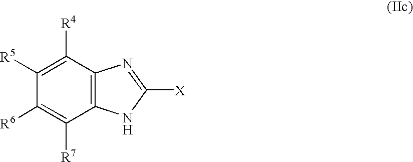

in which

R⁴, R⁵, R⁶ and R⁷ independently of one another represent hydrogen, fluorine, chlorine, bromine, iodine, cyano, nitro, straight-chain or branched alkyl having 1 to 8 carbon atoms, straight-chain or branched halogenoalkyl having 1 to 6 carbon atoms and 1 to 13 identical or different halogen atoms, straight-chain or branched alkoxy having 1 to 8 carbon atoms, straight-chain or branched halogenoalkoxy having 1 to 6 carbon atoms and 1 to 13 identical or different halogen atoms, straight-chain or branched alkylthio having 1 to 8 carbon atoms, straight-chain or branched halogenoalkylthio having 1 to 6 carbon atoms and 1 to 13 identical or different halogen atoms, straight-chain or branched alkylsulphinyl having 1 to 8 carbon atoms, straight-chain or branched halogenoalkylsulphinyl having 1 to 6 carbon atoms and 1 to 13 identical or different halogen atoms, straight-chain or branched alkylsulphonyl having 1 to 8 carbon atoms, straight-chain or branched halogenoalkylsulphonyl having 1 to 6 carbon atoms and 1 to 13 identical or different halogen atoms, cycloalkyl which has 3 to 6 carbon atoms and which is optionally monosubstituted to pentasubstituted by identical or different substituents from the series consisting of halogen and/or alkyl having 1 to 4 carbon atoms, or represent hydroxycarbonyl, alkylcarbonyl having 1 to 6 carbon atoms in the straight-chain or branched alkyl moiety, alkoxycarbonyl having 1 to 6 carbon atoms in the straight-chain or branched alkoxy moiety, cycloalkylcarbonyl having 3 to 6 carbon atoms in the cycloalkyl moiety, cycloalkoxycarbonyl having 3 to 6 carbon atoms in the cycloalkyl moiety, or represent -Z¹-R⁸ in which

R⁸ represents aryl having 6 to 10 carbon atoms, it being possible for each of these radicals to be monosubstituted to trisubstituted by identical or different substituents from the series consisting of halogen, cyano, nitro, alkyl having 1 to 4 carbon atoms, alkoxy having 1 to 4 carbon atoms, alkylthio having 1 to 4 carbon atoms, halogenoalkyl having 1 to 4 carbon atoms and 1 to 5 identical or different halogen atoms, halogenoalkoxy having 1 to 4 carbon atoms and 1 to 5 identical or different halogen atoms, halogenoalkylthio having 1 to 4 carbon atoms and 1 to 5 identical or different halogen atoms, alkylsulphinyl having 1 to 4 carbon atoms, alkylsulphonyl having 1 to 4 carbon atoms, halogenoalkylsulphinyl having 1 to 4 carbon atoms and 1 to 5 identical or different halogen atoms and/or halogenoalkylsulphonyl having 1 to 4 carbon atoms and 1 to 5 identical or different halogen atoms,

or

R⁸ also represents an unsaturated heterocyclyl radical with 5 or 6 ring members and 1 to 3 hetero atoms such as nitrogen, oxygen and/or sulphur, it being possible for these radicals to be monosubstituted to trisubstituted by identical or different substituents from the series consisting of halogen, alkyl having 1 to 4 carbon atoms, alkoxy having 1 to 4 carbon atoms, halogenoalkyl having 1 to 4 carbon atoms and 1 to 5 identical or different halogen atoms, halogenoalkoxy having 1 to 4 carbon atoms and 1 to 5 identical or different halogen atoms, alkoxycarbonyl having 1 to 3 carbon atoms in the alkoxy moiety, cycloalkyl having 3 to 6 carbon atoms, cyano and/or nitro,

and

Z¹ represents a direct bond and also —CH₂—, O, S, SO, SO₂ or CO, or

represents —CO—O—, where the oxygen atom is linked to R⁸, or

represents —SO₂—, where the sulphur atom is linked to R⁸, or

represents —S—CH₂—SO₂—O—, where the sulphur atom of the thio group is linked to R⁸,

or

R⁴ and R⁵, or R⁵ and R⁶, or R⁶ and R⁷ in each case together represent an alkylene chain which has 3 or 4 members and which is optionally monosubstituted to hexasubstituted by halogen, alkyl having 1 to 4 carbon atoms and/or halogenoalkyl having 1 to 4 carbon atoms and 1 to 9 halogen atoms and in which one or two (non-adjacent) carbon atoms can be replaced by oxygen atoms,

and

X represents chlorine or bromine.

Especially preferred starting materials for carrying out the process according to the invention are imidazole derivatives of the formula (II) in which

R¹ and R² independently of one another represent hydrogen, fluorine, chlorine, bromine, iodine, cyano, thiocyanato, nitro, formyl, carboxyl, carbamoyl, thiocarbamoyl, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl, ethylsulphonyl, trifluoromethyl, difluoromethoxy, trifluoromethoxy, difluoromethylthio, trifluoromethylthio, trifluoromethylsulphinyl or trifluoromethylsulphonyl, dimethylamino, diethylamino, acetyl, propionyl, acetyloxy, methoxycarbonyl, ethoxycarbonyl, methylsulphonyloxy, ethylsulphonyloxy, methoxyirminomethyl, ethoxyiminomethyl, methoxyiminoethyl and/or ethoxyirninoethyl or represent Z-R³ in which

Z represents a direct bond, an oxygen atom, or represents methanediyl, 1,1-ethanedyl, 1,2-ethanediyl, 1,1-, 1,2-, 1,3- or 2,2-propanediyl, 1,1-ethenediyl, 1,2-ethenediyl, ethinediyl,

-*Q-CQ-, -*CQ-Q-, —*CH₂-Q-, -*Q-CH₂—, -*CQ-Q-CH₂—, —*CH₂-Q-CQ-, -*Q-CQ-CH₂—, -*Q-CQ-Q-CH₂—, —S(O)ₙ—, —*CH₂—S(O)ₙ—, -CQ- or —*S(O)ₙ—-CH₂—,

where the atoms marked * are in each case linked to R³,

Q represents oxygen or sulphur,

n represents 0, 1 or 2

and

R³ represents phenyl or naphthyl, it being possible for each of these radicals to be monosubstituted to trisubstituted by identical or different substituents from the series consisting of fluorine, chlorine, bromine, cyano, nitro, formyl, carbamoyl, thiocarbamoyl, methyl, ethyl, n- or i-propyl, n-, i-, s- or t-butyl, methoxy, ethoxy, n- or i-propoxy, methylthio, ethylthio, n- or i-propylthio, methyl-sulphinyl, ethylsulphinyl, methylsulphonyl or ethylsulphonyl, trifluoromethyl, trifluoroethyl, difluoromethoxy, trifluoromethoxy, difluorochloromethoxy, trifluoroethoxy, difluoromethylthio, difluorochloromethylthio, trifluoromethyl-thio, trifluoromethylsulphinyl or trifluoromethylsulphonyl, dimethylamino, diethylamino, acetyl, propionyl, acetyloxy, methoxycarbonyl, ethoxycarbonyl, methylsulphonyloxy, ethylsulphonyloxy, methoximinomethyl, ethoximino-methyl, methoximinoethyl or ethoximinoethyl, cyclopropyl, cyclobutyl, cyclo-pentyl and/or cyclohexyl,

or else can be monosubstituted by in each case divalent methylenedioxy or ethylenedioxy, each of which is optionally monosubstituted to tetrasubstituted by fluorine and/or chlorine,

or

R³ represents an unsaturated heterocyclyl radical having 5 or 6 ring members and 1 to 3 hetero atoms such as nitrogen, oxygen and/or sulphur, it being possible for each of these radicals to be monosubstituted or disubstituted by identical or different substituents from the series consisting of fluorine, chlorine, bromine, methyl, ethyl, methoxy, ethoxy, halogenoalkyl having 1 or 2 carbon atoms and 1 to 5 fluorine, chlorine and/or bromine atoms, halogenoalkoxy having 1 or 2 carbon atoms and 1 to 5 fluorine, chlorine and/or bromine atoms, methoxycarbonyl, ethoxycarbonyl, cycloalkyl having 3 to 6 carbon atoms, cyano and/or nitro,

and

X represents chlorine or bromine.

Moreover, especially preferred starting materials for carrying out the process according to the invention are imidazole derivatives of the formula

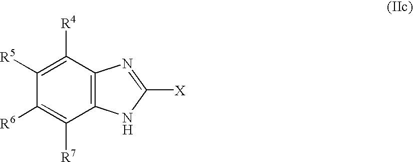

in which

R⁴, R⁵, R⁶ and R⁷ independently of one another represent hydrogen, fluorine, chlorine, bromine, iodine, cyano, nitro, straight-chain or branched alkyl having 1 to 6 carbon atoms, halogenoalkyl having 1 or 2 carbon atoms and 1 to 5 fluorine, chlorine and/or bromine atoms, straight-chain or branched alkoxy having 1 to 6 carbon atoms, halogenoalkoxy having 1 or 2 carbon atoms and 1 to 5 fluorine, chlorine and/or bromine atoms, straight-chain or branched alkylthio having 1 to 6 carbon atoms, halogenoalkylthio having 1 or 2 carbon atoms and 1 to 5 fluorine, chlorine and/or bromine atoms, straight-chain or branched alkylsulphinyl having 1 to 6 carbon atoms, halogenoalkylsulphinyl having 1 or 2 carbon atoms and 1 to 5 fluorine, chlorine and/or bromine atoms, straight-chain or branched alkylsulphonyl having 1 to 6 carbon atoms, halogenoalkylsulphonyl having 1 or 2 carbon atoms and 1 to 5 fluorine, chlorine and/or bromine atoms, cycloalkyl which has 3 to 6 carbon atoms and which is optionally monosubstituted to trisubstituted by identical or different substituents from the series consisting of fluorine, chlorine, bromine, methyl and/or ethyl, or represent hydroxycarbonyl, alkylcarbonyl having 1 to 4 carbon atoms in the straight-chain or branched alkyl moiety, alkoxycarbonyl having 1 to 4 carbon atoms in the straight-chain or branched alkoxy moiety, cycloalkylcarbonyl having 3 to 6 carbon atoms in the cycloalkyl moiety, cycloalkoxycarbonyl having 3 to 6 carbon atoms in the cycloalkyl moiety, or represent -Z¹-R⁸ in which

R⁸ represents phenyl which can be monosubstituted to trisubstituted by identical or different substituents from the series consisting of fluorine, chlorine, bromine, cyano, nitro, methyl, ethyl, methoxy, ethoxy, methylthio, ethylthio, halogenoalkyl having 1 or 2 carbon atoms and 1 to 5 fluorine, chlorine and/or bromine atoms, halogenoalkoxy having 1 or 2 carbon atoms and 1 to 5 fluorine, chlorine and/or bromine atoms, halogenoalkylthio having 1 or 2 carbon atoms and 1 to 5 fluorine, chlorine and/or bromine atoms, methyl-sulphinyl, ethylsulphinyl, methylsulphonyl, ethylsulphonyl, halogeno-alkylsulphinyl having 1 or 2 carbon atoms and 1 to 5 fluorine, chlorine and/or bromine atoms, and/or halogenoalkylsulphonyl having 1 or 2 carbon atoms and 1 to 5 fluorine, chlorine and/or bromine atoms, or

R⁸ represents an unsaturated heterocyclyl radical having 5 or 6 ring members and 1 to 3 hetero atoms such as nitrogen, oxygen and/or sulphur, it being possible for each of these radicals to be monosubstituted or disubstituted by identical or different substituents from the series consisting of fluorine, chlorine, bromine, methyl, ethyl, methoxy, ethoxy, halogenoalkyl having 1 or 2 carbon atoms and 1 to 5 fluorine, chlorine and/or bromine atoms, halogenoalkoxy having 1 or 2 carbon atoms and 1 to 5 fluorine, chlorine and/or bromine atoms, methoxycarbonyl, ethoxycarbonyl, cycloalkyl having 3 to 6 carbon atoms, cyano and/or nitro,

and

Z¹ represents a direct bond and also CH₂, O, S, SO, SO₂ or CO, or

represents —CO—O—, where the oxygen atom is linked to R⁸, or

represents —SO₂—O—, where the sulphur atom is linked to R⁸, or

represents —S—CH₂—SO₂—, where the sulphur atom of the thio group is linked to R⁸,

or

R⁴ and R⁵, or R⁵ and R⁶, or R⁶ and R⁷ in each case together represent an alkylene chain with 3 or 4 members which is optionally monosubstituted to hexasubstituted by fluorine, chlorine, methyl and/or trifluoromethyl and in which one or two (non-adjacent) carbon atoms can be replaced by oxygen,

and

X represents chlorine or bromine.

Very especially preferred starting materials for carrying out the process according to the invention are imidazole derivatives of the formula

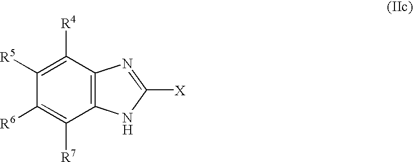

in which

R⁴, R⁵, R⁶ and R⁷ independently of one another represent hydrogen, fluorine, chlorine, bromine, cyano, nitro, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl, ethylsulphonyl, trifluoromethyl, difluoromethoxy, trifluoromethoxy, difluoromethylthio, trifluoromethylthio, trifluoromethylsulphinyl, trifluoromethylsulphonyl, acetyl, acetyloxy, methoxycarbonyl, ethoxycarbonyl, cyclopropyl, cyclohexyl or Z¹-R⁸

in which

R⁸ represents phenyl which can be monosubstituted to trisubstituted by identical or different substituents from the series consisting of fluorine, chlorine, bromine, cyano or nitro, or represents methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl, ethylsulphonyl, trifluoromethyl, difluoromethoxy, trifluoromethoxy, difluoromethylthio, trifluoromethylthio, difluoromethylsulphinyl and/or trifluoromethylsulphonyl,

or

R⁸ represents pyrrolyl, furyl, thienyl, pyrazolyl, imidazolyl, thiazolyl, isothiazolyl, oxazolyl, isoxazolyl, oxadiazolyl, thiadiazolyl, 1,2,3-triazinyl, 1,2,4-triazinyl, 1,3,5-triazinyl, pyridinyl, pyrimidinyl, pyrazinyl or pyridazinyl, it being possible for each of these radicals to be monosubstituted or disubstituted by identical or different substituents from the series consisting of fluorine, chlorine, methyl, methoxy, trifluoromethyl, trifluoromethoxy and/or trifluoromethylthio,

and

Z¹ represents a direct bond and also CH₂, O, S, SO, SO₂ or CO, or

represents —CO—O—, where the oxygen atom is linked to R⁸, or

represents —SO₂—O—, where the sulphur atom is linked to R⁸, or

represents —S—CH₂—SO₂—, where the sulphur atom of the thio group is linked to R⁸,

or

R⁴ and R⁵, or R⁵ and R⁶, or R⁶ and R⁷ in each case together represent the groups

—CF₂—O—CF₂—, —O—CF₂—O—, —O—CF₂—CHF—O—, —O—CHF—CHF—O—, —CF₂—CF₂—O—, —O—CF₂—CFCl—O— or —O—CFCl—CFCl—O—,

and

X represents chlorine or bromine.

The imidazole derivatives of the formula (II) are known or can be prepared by known processes (cf. WO 97-06 171, WO 97-47 620, EP-A 0 893 445, DE-A 198 29 740 and JP-A 54-90 175).

Formula (III) provides a general definition of the sulphonyl chlorides furthermore required as starting materials for carrying out the process according to the invention. Preferred compounds of the formula (III) are those in which

R represents straight-chain or branched alkyl having 1 to 4 carbon atoms, straight-chain or branched halogenoalkyl having 1 to 4 carbon atoms and 1 to 5 identical or different halogen atoms, straight-chain or branched alkoxy having 1 to 4 carbon atoms, straight-chain or branched halogenoalkoxy having 1 to 4 carbon atoms and 1 to 5 identical or different halogen atoms, straight-chain or branched alkylthio having 1 to 4 carbon atoms, straight-chain or branched halogenoalkylthio having 1 to 4 carbon atoms and 1 to 5 identical or different halogen atoms, straight-chain or branched alkenyl having 2 to 4 carbon atoms, straight-chain or branched halogenoalkenyl having 2 to 4 carbon atoms and 1 to 5 identical or different halogen atoms, straight-chain or branched alkenyloxy having 2 to 4 carbon atoms, straight-chain or branched alkenylthio having 2 to 4 carbon atoms, straight-chain or branched alkinyl having 2 to 4 carbon atoms, straight-chain or branched alkinyloxy having 2 to 4 carbon atoms, straight-chain or branched alkinylthio having 2 to 4 carbon atoms, alkylamino having 1 to 4 carbon atoms, dialkyl having 1 to 4 carbon atoms in each alkyl moiety,

or

R represents phenyl, phenoxy or phenylthio, it being possible for each of these radicals to be monosubstituted to trisubstituted by identical or different substituents from the series consisting of fluorine, chlorine, bromine, alkyl having 1 to 4 carbon atoms, halogenoalkyl having 1 to 4 carbon atoms and 1 to 5 fluorine, chlorine and/or bromine atoms, alkoxy having 1 to 4 carbon atoms and/or halogenoalkoxy having 1 to 4 carbon atoms and 1 to 5 fluorine, chlorine and/or bromine atoms,

or

R represents cycloalkyl having 3 to 7 carbon atoms, cycloalkyloxy having 3 to 7 carbon atoms, cycloalkylthio having 3 to 7 carbon atoms, cycloalkylamino having 3 to 7 carbon atoms, pyrrolidinyl, piperidinyl, or morpholinyl, it being possible for each of these abovementioned radicals to be monosubstituted to trisubstituted by identical or different substituents from the series consisting of fluorine, chlorine, bromine, alkyl having 1 to 4 carbon atoms and/or halogenoalkyl having 1 to 4 carbon atoms and 1 to 5 fluorine, chlorine and/or bromine atoms,

or

R represents an unsaturated heterocyclyl radical having 5 or 6 ring members and 1 to 3 hetero atoms such as nitrogen, oxygen and/or sulphur, it being possible for these radicals to be monosubstituted to trisubstituted by identical or different substituents from the series consisting of halogen, cyano, nitro, hydroxyl, amino, formyl, carboxyl, carbamoyl, thiocarbamoyl, alkyl having 1 to 4 carbon atoms, alkoxy having 1 to 4 carbon atoms, halogenoalkyl having 1 to 4 carbon atoms and 1 to 5 identical or different halogen atoms, halogenoalkoxy having 1 to 4 carbon atoms and 1 to 5 identical or different halogen atoms, alkoxycarbonyl having 1 to 4 carbon atoms in the alkoxy moiety, cycloalkyl having 3 to 6 carbon atoms, halogenoalkylsulphinyl having 1 to 4 carbon atoms and 1 to 5 identical or different halogen atoms, halogenoalkylsulphonyl having 1 to 4 carbon atoms and 1 to 5 identical or different halogen atoms, alkylamino having 1 to 4 carbon atoms, hydroxyalkylamino having 1 to 4 carbon atoms, dialkylamino having 1 to 4 carbon atoms in each alkyl group, alkylcarbonyl having 1 to 4 carbon atoms in the alkyl moiety, hydroximinoalkyl having 1 to 4 carbon atoms in the alkyl moiety, alkoximinoalkyl having 1 to 4 carbon atoms in the alkoxy moiety and 1 to 4 carbon atoms in the alkyl moiety, alkylcarbonyloxy having 1 to 4 carbon atoms in the alkyl moiety and/or halogenoalkylcarbonyloxy having 1 to 4 carbon atoms in the halogenoalkyl group and 1 to 5 identical or different halogen atoms.

Especially preferred starting materials are sulphonyl chlorides of the formula (III), in which

R represents methyl, ethyl, n- or i-propyl, n-, i-, s- or t-butyl, trifluoromethyl, trifluoroethyl, methoxy, ethoxy, n- or i-propoxy, n-, i-, s- or t-butoxy, di-fluoromethoxy, trifluoromethoxy, difluorochloromethoxy, trifluoroethoxy, methylthio, ethylthio, n- or i-propylthio, difluoromethylthio, trifluoromethyl-thio, difluorochloromethylthio, allyl, n- or s-butenyl; allyloxy, n- or s-butenyloxy; allylthio, n- or s-butenylthio; propargyl, n- or s-butinyl; propargyloxy; propargylthio; methylamino, ethylamino, n- or i-propylamino, n-, i-, s- or t-butylamino; dimethylamino, diethylamino, di-n- or i-propylamino, methylethylamino, methyl-n- or i-propylamino, or

R represents phenyl, phenoxy or phenylthio, it being possible for each of these radicals to be monosubstituted to trisubstituted by identical or different substituents from the series consisting of fluorine, chlorine, bromine, methyl, ethyl, n- or i-propyl, methoxy, ethoxy, n- or i-propoxy, trifluoromethyl and/or trifluoromethoxy; or

R represents cyclopropyl, cyclopentyl, cyclohexyl, cyclopropyloxy, cyclopentyloxy, cyclohexyloxy, cyclopropylthio, cyclopentylthio, cyclohexylthio, cyclopropylamino, cyclopentylamino, cyclohexylamino, 1-pyrrolidinyl, 1-piperidinyl or 1-morpholinyl, it being possible for each of these radicals to be monosubstituted to trisubstituted by identical or different substituents from the series consisting of fluorine, chlorine, bromine, methyl, ethyl, n- or i-propyl and/or trifluoromethyl, or

R represents an unsaturated heterocyclyl radical having 5 or 6 ring members and 1 to 3 hetero atoms such as nitrogen, oxygen and/or sulphur, it being possible for each of these radicals to be monosubstituted or disubstituted by identical or different substituents from the series consisting of fluorine, chlorine, bromine, cyano, nitro, hydroxyl, amino, formyl, carboxyl, carbamoyl, thiocarbamoyl, alkyl having 1 to 4 carbon atoms, alkoxy having 1 to 4 carbon atoms, halogenoalkyl having 1 or 2 carbon atoms and 1 to 5 fluorine, chlorine and/or bromine atoms, halogenoalkoxy having 1 or 2 carbon atoms and 1 to 5 fluorine, chlorine and/or bromine atoms, alkoxycarbonyl having 1 or 2 carbon atoms in the alkoxy moiety, cycloalkyl having 3 to 6 carbon atoms, halogeno-alkylsulphinyl having 1 or 2 carbon atoms and 1 to 5 fluorine, chlorine and/or bromine atoms, halogenoalkylsulphonyl having 1 or 2 carbon atoms and 1 to 5 fluorine, chlorine and/or bromine atoms, alkylamino having 1 or 2 carbon atoms, hydroxyalkylamino having 1 or 2 carbon atoms, dialkylamino having 1 or 2 carbon atoms in each alkyl group, alkylcarbonyl having 1 or 2 carbon atoms in the alkyl moiety, hydroximinoalkyl having 1 or 2 carbon atoms in the alkyl moiety, alkoximinoalkyl having 1 or 2 carbon atoms in the alkoxy moiety and 1 or 2 carbon atoms in the alkyl moiety, alkylcarbonyloxy having 1 or 2 carbon atoms in the alkyl group and/or halogenoalkylcarbonyloxy having 1 or 2 carbon atoms in the halogenoalkyl group and 1 to 5 fluorine, chlorine and/or bromine atoms.

Very especially preferred starting materials are sulphonyl chlorides of the formula (III) in which

R represents methyl, ethyl, n- or i-propyl, n-, i-, s- or t-butyl, trifluoromethyl, trifluoroethyl, methoxy, ethoxy, n- or i-propoxy, n-, i-, s- or t-butoxy, difluoromethoxy, trifluoromethoxy, difluorochloromethoxy, trifluoroethoxy, methylthio, ethylthio, n- or i-propylthio, difluoromethylthio, trifluoromethylthio, difluorochloromethylthio, allyl, n- or s-butenyl; allyloxy, n- or s-butenyloxy; allylthio, n- or s-butenylthio; propargyl, n- or s-butinyl; propargyloxy; propargylthio; methylamino, ethylamino, n- or i-propylamino, n-, i-, s- or t-butylamino; dimethylamino, diethylamino, di-n- or i-propylamino, methylethylamino, methyl-n- or i-propylamino; or

R represents phenyl, phenoxy or phenylthio, it being possible for each of these radicals to be monosubstituted to trisubstituted by identical or different substituents from the series consisting of fluorine, chlorine, bromine, methyl, ethyl, n- or i-propyl, methoxy, ethoxy, n- or i-propoxy, trifluoromethyl and/or trifluoromethoxy; or

R represents cyclopropyl, cyclopentyl, cyclohexyl, cyclopropyloxy, cyclopentyloxy, cyclohexyloxy, cyclopropylthio, cyclopentylthio, cyclohexylthio, cyclopropylamino, cyclopentylamino, cyclohexylamino, 1-pyrrolidinyl, 1-piperidinyl or 1-morpholinyl, it being possible for each of these radicals to be monosubstituted to trisubstituted by identical or different substituents from the series consisting of fluorine, chlorine, bromine, methyl, ethyl, n- or i-propyl and/or trifluoromethyl,

or

R represents pyrrolyl, furyl, thienyl, pyrazolyl, imidazolyl, thiazolyl, isothiazolyl, oxazolyl, isoxazolyl, oxadiazolyl, thiadiazolyl, 1,2,3-triazinyl, 1,2,4-triazinyl, 1,3,5-triazinyl, pyridinyl, pyrimidinyl, pyrazinyl or pyridazinyl, it being possible for each of these radicals to be monosubstituted or disubstituted by identical or different substituents from the series consisting of fluorine, chlorine, bromine, cyano, nitro, amino, hydroxyl, formyl, carboxyl, carbamoyl, thiocarbarnoyl, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl, ethylsulphonyl, trifluoromethyl, difluoromethoxy, trifluoromethoxy, difluoromethylthio, trifluoromethylthio, trifluoromethylsulphinyl or trifluoromethylsulphonyl, methylamino, ethylaniino, n- or i-propylamino, dimethylamino, diethylamino, acetyl, propionyl, acetyloxy, methoxycarbonyl, ethoxycarbonyl, methylsulphonyloxy, ethylsulphonyloxy, hydroxyiminomethyl, hydroxyiminoethyl, methoxyiminomethyl, ethoxyiminomethyl, methoxyiminoethyl and/or ethoxyiminoethyl.

The sulphonyl chlorides of the formula (III) are known or can be prepared by known methods (cf. WO 97-06 171, WO 97-47 620 and DE-A 198 29 740).

Suitable acid binders for carrying out the process according to the invention are, preferably, the hydroxides, acetates, carbonates or hydrogen carbonates of alkaline earth metals or alkali metals. The following may be mentioned as examples: sodium hydroxide, potassium hydroxide, sodium acetate, potassium acetate, calcium acetate, sodium carbonate, potassium carbonate, potassium hydrogen carbonate. The following can furthermore be used: ammonium compounds and organic bases such as ammonium acetate or ammonium carbonate, or tertiary amines such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable diluents for carrying out the process according to the invention are all customary organic solvents which are sparingly miscible with water. The following can preferably be used: aliphatic, alicyclic or aromatic hydrocarbons such as petroleum ether, hexane, heptane, cyclohexane, methylcyclohexane, benzene, toluene, xylene or decalin; furthermore halogenated hydrocarbons such as chlorobenzene, dichlorobenzene, dichloromethane, chloroform, tetrachloromethane, dichloroethane or trichloroethane; furthermore ethers such as diethyl ether, diisopropyl ether, methyl t-butyl ether, methyl t-amyl ether, dioxane, tetrahydrofuran, 1,2-dimethoxyethane, 1,2-diethoxyethane or anisole; and esters such as methyl acetate or ethyl acetate.

Suitable phase transfer catalysts for carrying out the process according to the invention are all reaction accelerators which can conventionally be employed for this purpose. The following can preferably be used: tetrabutylammonium iodide, tetrabutylammonium bromide, tetrabutylammonium chloride, tributyl-methylphosphonium bromide, trimethyl-C₁₃/C₁₅-alkylammonium chloride, trimethyl-C₁₃/C₁₅-alkylammonium bromide, dibenzyl-dimethyl-ammoniummethyl sulphate, dimethyl-C₁₂/C₁₄-alkyl-benzylammonium chloride, dimethyl-C₁₂/C₁₄-alkyl-benzylammonium bromide, tetrabutylammonium hydroxide, triethylbenzylammonium chloride, methyl-trioctylammonium chloride, trimethylbenzyl-ammonium chloride, 15-crown-5, 18-crown-6 and tris-[2-(2-methoxyethoxy)-ethyl]-amine.

When carrying out the process according to the invention, the reaction temperatures can be varied within a substantial range. In general, the process is carried out at temperatures between 0° C. and 130° C., preferably between 20° C. and 50° C.

The process according to the invention is generally carried out under atmospheric pressure. However, it may also be carried out under elevated or reduced pressure.

When carrying out the process according to the invention, an equivalent amount or else an excess of sulphonyl chloride of the formula (III) and an equivalent amount or else an excess of acid binder are generally employed per mole of imidazole derivative of the formula (II). Work-up is carried out by customary methods. In general, a procedure is followed in which the organic phase is separated off, the aqueous phase is extracted with an organic solvent which is sparingly miscible with water, and the combined organic phases are, if appropriate, washed and then dried and concentrated. However, it is also possible to work up the mixture without addition of water by removing any solids of the formula (I) by filtration. Impurities which may still be present can be removed by customary methods, for example by recrystallization or by chromatography.

The sulphonyl-imidazole derivatives of the formula (I) which can be prepared by the process according to the invention are known as active substances with microbicidal, in particular fungicidal, properties (cf. WO 97-06 171, WO 97-47 620 and DE-A 198 29 740).

The procedure of the process according to the invention is illustrated by the examples which follow.

### PREPARATION EXAMPLES

### Example 1

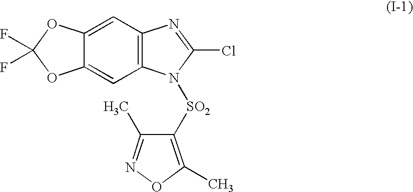

7.7 g (39 mmol) of 3,5-dimethyl-isoxazole-4-sulphonyl chloride, 1.0 g (3.0 mmol) of tetra-(n-butyl)-ammonium bromide, 5.3 g (39 mmol) of potassium carbonate and 100 ml of water are added at room temperature to a stirred mixture of 9.0 g (36 mmol) of 2-chloro-6,6-difluoro-[1,3]-dioxolo-[4,5-f]-1,3-benzimidazole and 100 ml of toluene. When the addition has ended, the reaction mixture is stirred for a further 4 hours at room temperature. The organic phase is subsequently separated off. The aqueous phase is extracted with toluene. The combined organic phases are washed in succession with 5% aqueous hydrochloric acid and aqueous sodium hydrogen carbonate solution, then dried over sodium sulphate and concentrated under reduced pressure. This gives 14.5 g of a colourless solid which, according to HPLC (ISTD), consists to 97.7% of 1-(3,5-dimethyl-isoxazole-4-sulphonyl)-2-chloro-6,6-difluoro-[1,3]-dioxolo-[4,5-f]-benzimidazole. Accordingly, the yield is calculated as 93% of theory.

### Example 2

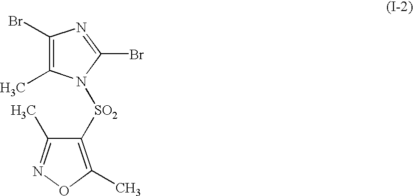

5.3 g (26.9 mmol) of 3,5-dimethyl-isoxazole-4-sulphonyl chloride, 0.2 g (0.6 mmol) of tetra-(n-butyl)-ammonium bromide, 3.7 g (26.9 mmol) of potassium carbonate and 25 ml of water are added at room temperature to a stirred mixture of 6.0 g (24.4 mmol) of 2,4-dibromo-5-methyl-imidazole and 25 ml of toluene. After the addition has ended, the reaction mixture is stirred for a further 4.5 hours at 50° C. The organic phase is subsequently separated off. The aqueous phase is extracted with toluene. The combined organic phases are dried over sodium sulphate and then concentrated under reduced pressure. This gives 8.4 g of a solid which, according to HPLC, consists to 86.8% of 1-(3,5-dimethyl-isoxazol-4-yl-sulphonyl)-2,4-dibromo-5-methyl-imidazole. Accordingly, the yield is calculated as 75% of theory.

### Example 3

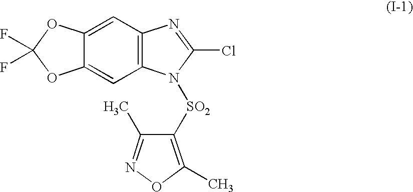

A mixture of 40 kg of 2-chloro-6,6-difluoro-[1,3]-dioxolo-[4,5-f]-1,3-benzimidazole (purity: 94%), 5.5 kg of tetrabutylammonium bromide and 22 kg of triethylamine is added with stirring at room temperature to 170 kg of water. 140 kg of toluene are then added to the reaction mixture. 95 kg of 3,5-dimethyl-isoxazole-4-sulphonyl chloride in toluene are subsequently added in the course of 5 hours, during which process the temperature of the reaction mixture is kept to 20 to 30° C. The proportioning pump is then washed with 2 kg of toluene and the wash liquid is added to the reaction mixture. After stirring has been continued for three hours at temperatures between 20° C. and 30° C., the reaction mixture is filtered. The filtration residue is washed three times with in each case 200 kg of water and then dried at 40 to 50° C. under reduced pressure.

In this manner, a total of 119 kg of 2-chloro-6,6-difluoro-[b 1,3]-dioxolo-[4,5-f]-1,3-benzimidazole (purity: 94%) are reacted in three passes.

The three batches yield a total of 132 kg of a product which consists to 94% of 1-(3,5-dimethyl-isoxazole-4-sulphonyl)-2-chloro-6,6-difluoro-[1,3]-dioxolo-[4,5-f]-benzimidazole. Accordingly, the yield is calculated as 66% of theory.

The mother liquor contains a further 38 kg of 1-(3,5-dimethyl-isoxazole-4-sulphonyl)-2-chloro-6,6-difluoro-[1,3]-dioxolo-[4,5-f]-benzimidazole, which corresponds to an additional yield of 20% of theory.

### Comparative Example A

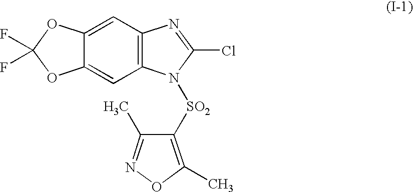

81.6 g (0.6 mol) of pulverulent potassium carbonate are added at room temperature to a stirred mixture of 96.6 g (0.4 mol) of 2-chloro-6,6-difluoro-[1,3]-dioxolo-[4,5-f]-1,3-benzimidazole and 600 ml of acetonitrile, and the mixture is stirred for 10 minutes at room temperature. 79.2 g (0.4 mol) of 3,5-dimethyl-isoxazole-4-sulphonyl chloride are subsequently added, and stirring is continued for 20 hours at room temperature. The reaction mixture is poured into 2 litres of water. The resulting mixture is extracted three times using in each case 500 ml of methylene chloride. The combined organic phases are dried over sodium sulphate and concentrated under reduced pressure. The residue which remains is chromatographed on silica gel with methylene chloride as mobile phase. This gives 117 g (75% of theory) of 1-(3,5-dimethyl-isoxazole-4-sulphonyl)-2-chloro-6,6-difluoro-[1,3]dioxolo-[4,5-f]-benz-imidazole in the form of a colourless solid of melting point 128 to 131° C.

### Comparative Example B

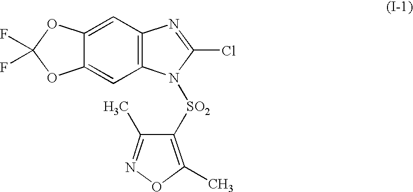

7.7 g (39 mmol) of 3,5-dimethyl-isoxazole-4-sulphonyl chloride, 5.3 g (39 mmol) of potassium carbonate and 100 ml of water are added at room temperature to a stirred mixture of 9.0 g (36 mmol) of 2-chloro-6,6-difluoro-[1,3]-dioxolo-[4,5-f]-1,3-benz-imidazole and 100 ml of toluene. After the addition has ended, the reaction mixture is stirred for a further 3 hours at room temperature. Then, a sample is removed and examined by thin-layer chromatography. No reaction to give 1-(3,5-dimethyl-isoxazole-4-sulphonyl)-2-chloro-6,6-difluoro-[1,3]-dioxolo-[4,5-f]-benzimidazole can be detected.

1. Process for the preparation of sulphonyl-imidazole derivatives of the formula 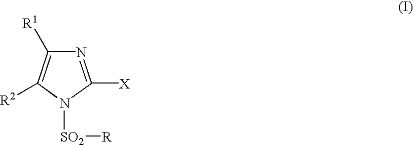
in which
R¹ and R² are identical or different and independently of one another represent hydrogen, halogen, cyano, thiocyanato, nitro, formyl, carboxyl, carbamoyl, thiocarbamoyl, alkyl, alkoxy, alkylthio, alkylsulphinyl, alkylsulphonyl, alkenyl, alkenyloxy, halogenoalkyl, halogenoalkoxy, halogenoalkylthio, halogenoalkylsulphinyl, halogenoalkylsulphonyl, halogenoalkenyl, halogenoalkenyloxy, alkylcarbonyl, alkylcarbonyloxy, alkoxycarbonyl, alkylsulphonyloxy, alkoximinoalkyl, cycloalkyl, dialkylamino or Z-R³ where
Z represents a direct bond, an oxygen atom, alkanediyl, alkenediyl, alkinediyl, -*Q-CQ-, -*CQ-Q-, —*CH₂-Q-, -Q*-CH₂—, -*CQ-Q-CH₂—, —*CH₂-Q-CQ-, -*Q-CQ-CH₂—, -*Q-CQ-Q-CH₂—, —S(O)ₙ—, —*CH₂—S(O)ₙ—, -CQ- or —*S(O)ₙ—CH₂—, in which
Q represents oxygen or sulphur and the atoms marked (*) are in each case linked to R³,
n represents 0, 1 or 2 and
R³ represents optionally substituted aryl or optionally substituted heterocyclyl,
or
R¹ and R² together represent a divalent radical of the formula 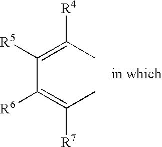
R⁴, R⁵, R⁶ and R⁷ independently of one another represent hydrogen, halogen, cyano, nitro, alkyl, halogenoalkyl, alkoxy, halogenoalkoxy, alkylthio, halogenoalkylthio, alkylsulphinyl, halogenoalkylsulphinyl, alkylsulphonyl, halogenoalkylsulphonyl, optionally substituted cycloalkyl, hydroxycarbonyl, alkylcarbonyl, alkoxycarbonyl, cycloalkylcarbonyl, cycloalkoxycarbonyl or -Z¹-R⁸, in which
R⁸ represents optionally substituted aryl or optionally substituted heterocyclyl and
Z¹ represents a direct bond, or represents —CH₂—, O, S, SO, SO₂, or CO, or
represents —CO—O—, where the oxygen atom is linked to R⁸, or
represents —SO₂—O—, where the sulphur atom is linked to R⁸, or
represents —S—CH₂—SO₂—, where the sulphur atom of the thio group is linked to R⁸,
or
R⁴ and R⁵, or R⁵ and R⁶, or R⁶ and R⁷ in each case together represent an optionally substituted alkylene chain with 3 or 4 members in which one or two (non-adjacent) carbon atoms can be replaced by oxygen atoms,
R represents alkyl, halogenoalkyl, alkoxy, halogenoalkoxy, alkylthio, halogenoalkylthio, alkenyl, halogenoalkenyl, alkenyloxy, alkenylthio, alkinyl, alkinyloxy, alkinylthio, alkylamino, dialkylamino, optionally substituted aryl, optionally substituted aryloxy, optionally substituted arylthio, optionally substituted cycloalkyl, optionally substituted cycloalkyloxy, optionally substituted cycloalkylthio, optionally substituted cycloalkylamino, optionally substituted di-cycloalkyl-amino or represents an optionally substituted, saturated or unsaturated heterocyclic radical
and
X represents fluorine, chlorine or bromine, characterized in that imidazole derivatives of the formula 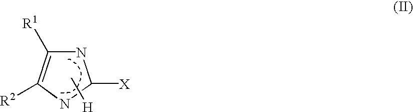
in which
R¹, R² and X are as defined above, are reacted with sulphonyl chlorides of the formula
Cl—SO₂—-R (III)
in which R is as defined above,
in the presence of an acid binder, of an organic diluent which is sparingly miscible with water and in the presence of water and a phase transfer catalyst.

2. Process according to claim 1, characterized in that the imidazole derivative employed is 2-chloro-6,6-difluoro-[1,3]-dioxolo-[4,5-f]-benzimidazole of the formula

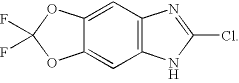

3. Process according to claim 1, characterized in that the sulphonyl chloride employed is 3,5-dimethyl-isoxazole-4-sulphonyl chloride, of the formula

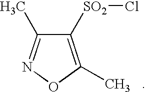

4. Process according to claim 1, characterized in that the reaction is carried out at temperatures between 0° C. and 130° C.
